(19) United States
(12) Patent Application Publication
(10) Pub. No.: US 2019/0091057 A1
(43) Pub. Date: Mar. 28, 2019

(54) Contour Bladder Bandage for Treatment of Pressure Ulcers

(71) Applicant: George Anthony Elizarraras, (US)

(72) Inventor: George Anthony Elizarraras, Spring, TX (US)

(21) Appl. No.: 15/713,650

(22) Filed: Sep. 23, 2017

(51) Int. Cl. A61F 5/34

(52) U.S. Cl. CPC A61F 5/34

(57) ABSTRACT

A medical bandage for the treatment of pressure ulcers as part of wound care management, consisting of a flexible bladder cushion, conforming to the body shape to relieve pressure by redistributing the concentration forces as leading causes of wound(s) and allowing for ventilation for increasing healing. The bandage incorporates an absorbent padding and an adhesive flange.

### BACKGROUND

Individuals who are incumbent to prolonged immobility, sitting or lying, or experiencing frictional forces for extended periods are prone to pressure ulcer wounds (also known as decubitus ulcers or bedsores). Such localized injuries result in necrosis to the skin and underlying tissue and are typical at locations of the body where bony prominence exists when the skin breaks down at points of pressure or combinations of conditions with frictional forces (rubbing, chaffing, shear) or tissue strains or conditions of maceration of the skin. Additional complications can arise in perennial bedridden patients due to malnutrition, diabetes, vascular or neurological disorders, age, or unfavorable conditions leading to wound exacerbation or antagonizing of skin or by acts of infection.

Focus of treatment is rooted in early detection and preventative measures, but once developed, ulcers can be painful and very difficult to treat because of underlying circulatory problems. Tissue recovery can be slow and treatment over an extensive time can be painful.

Preventative treatment and therapies like conventional cushioning bladder devices, inflatable pillows, support cushions, air fluidized beds/pads, air permeable mattresses, frequency modulation pressure pads, inhibiting hammock devices and other devices all have an inherent drawback impeding comfort or preventing proper resolution to the underlying conditions associated with skin accessibility to air or surface contact friction.

Conventional dressings, such as gaze, bandage, wet dressings (hydrogel, hydrocolloids) may suffocate the wound and retard healing or scabbing. Many bandages are planar and do not conform to body curvature; and creates further complications by layers and folds of the dressing. In some circumstances, the dressing may further antagonize the wound and become a source of irritation that worsens the wound.

While medical care and proprietary therapies (e.g. Negative Pressure Wound Therapy) for the chronic wounds may suffice as an appropriate approach for some wounds they are sometimes expensive, elaborate, componentized, and not readily available as a consumable product. Likewise, wound dressing and padding is inadequate in contoured regions of the body, such as the sacrum and coccyx region. Unsatisfactory techniques or device utilities fail to address the combined underlying issues of vascular circulation and debridement management; to which there is a need for improved devices and solutions.

### BRIEF SUMMARY OF INVENTION

To address the above mentioned problems, the medical utility discloses improvement in the unique design of the article, the function of article, and method of application to promote accelerated healing recovery and comfort. This medical utility discloses a contour bandage for the treatment of pressure ulcers as part of wound care management. The bandage consist of a flexible bladder cushion body arranged to shield the peripheral of the wound while relieving pressure and friction forces by redistributing the forces causing the wound; also allowing air circulation in the vicinity of the wound.

The article emphasizes design to provide pressure relief to the wound vicinity; and allow vascular circulation. The bladder conforms to the body shape for the assurance of relief. Particular to anatomical sites complicated by areas of concave contour, such as the coccyx (tailbone).

The article promotes separation of wound from contact surfaces to allow air ventilation that can promote healing, reduce potential of skin maceration, promote preparation for wound cleaning, or preparation of the vicinity for the debridement of damaged or infected tissue.

The article incorporates a protective sterile absorbent (or hydrophilic) and flexible textile layer or padding. The padding allows for absorption of exuding wound fluids or for the application of balm, cream, or other medication(s).

The article incorporates a flexible adhesive flange (border) for application of the bandage. The flange is designed to improve adhesion to irregular shaped surfaces.

The article allows for dressing of the wound with consideration to contoured body regions, such as the coccyx region.

Additional embodiments allow for fluid filled pods or gel filler for heating or cooling. In another embodiment the bladder chambers are separated to allow for different pressures in different locations. In another embodiment the bladder and padding is heart shaped or tulip shaped or tear drop shaped to better fit particular wounds common with coccyx wounds.

### BRIEF DESCRIPTION OF DIAGRAMS/DRAWING

FIG. 1 is a sectional view of the bladder bandage

FIG. 2 is a schematic plan view illustrating common risk prone body portions for application of present invention

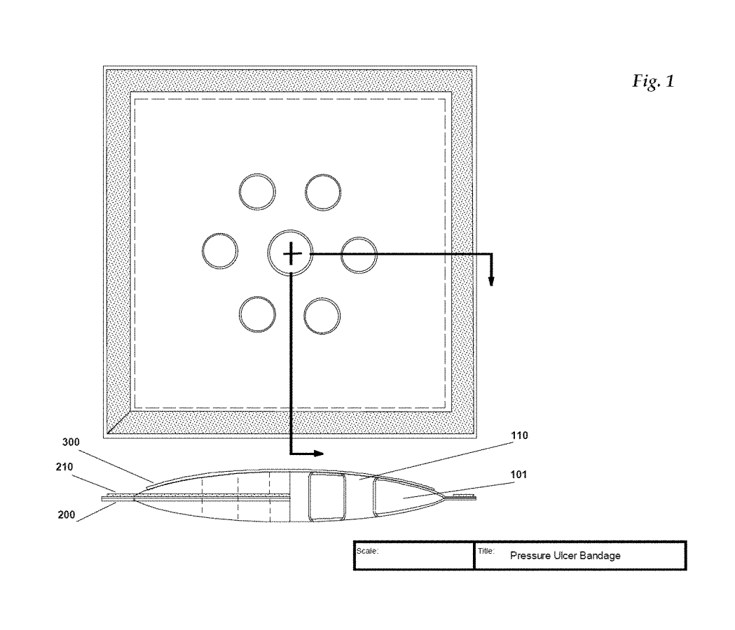

### DETAILED DESCRIPTION

Those of ordinary skill in the art will readily recognize many alternative embodiments, but detailed description of preferred embodiments of the present invention, the scope of which is limited only by the claims appended hereto. Illustrations are for typical preferred application of the present invention for treatment of pressure ulcer, or other wound, at location of wound.

The article described as a bandage, according to the present invention, which is intended for use by the transfer and redistribute of pressure concentration resulting in friction points or skin shear movement; and invention is applied to a patient's body member, such as at the sacrum or coccyx region 500. Areas of risk emphasize locations where bone is close to skin in contact with external surfaces that may include, but not limited to, sacrum and coccyx and ischium 500, occiput 501, scapula 502, calcaneus and lateral malleolus 503, knee 504, trochanter 505, elbow, iliac crest, or spinous process. The bandage redistributes weight to reduce pressure concentration and lessen frictional forces at source of wound, without hindrance to blood circulation. The bandage is comprised of a bladder chamber body 101, ventilation ports 110, adhesive flange 200, and absorbent padding 300.

The bandage assembly is made from soft elastomeric material or like gas-tight impermeable material or resilient textile for the formation of a bladder chamber body or fluid pocket 101, or plurality of. Considerations are made for film-joining methods and welding processes for thermoplastic films; from RF welding, dielectric sealing, ultrasonic welding, thermal sealing, induction welding, solvent bonding, etc. The bladder of the bandage acts as the cushioning body and is intended to be filled and pressurized with gas, liquid, gel, or fluid for distribution of point concentrations associated with ulcer wound development.

The bladder body 200 in preferred embodiment is pressurized to a pressure corresponding to the forces associated with patient's body weight at the concentration point. In other embodiments considerations are made to recognize altering of bladder pressure with fill-ports; which may be useful where alterations are needed to multiple arrayed independent bladder chambers.

The bandage is characterized with ventilation ports 110, an open passage through the coronal plane along the posterior anterior axis. The port is of dimension to allow ventilation of the wound area, thus encouraging wound healing with air exposure. Consideration is given so that during pressure redistribution the ports do not collapse to choke off ventilation.

The bandage is of shape corresponding to comfort, preferred embodiment of rectangular shape and deemed appropriate for sitting or lying. Alternative embodiments consider shapes of body and flange for comfort and performance optimization.

The bandage is characterized with a flange 200 around the peripheral of the bladder body for secure attachment of bandage, as positioned over the wound with flange encircling the wound area. The flange, in preferred embodiment, is part of the same flexible material as the posterior or anterior material. The flange, in preferred embodiment, has an adhesive seal 210 comprised of an adhesive strip peripherally attached to the outer layer of the flange, and for secure fixing of bandage in desired location. Alternative embodiments consider films of differing pliability, permeability, and thickness for improved comfort. Likewise, alternative embodiments considers the adhesive strip as protected by a peelable and disposable apron.

The bandage consists of absorbent and permeable padding 300 as part of dressing application over wound vicinity. Padding is without hindrance to the benefits promoted by ventilation ports 110. The padding can be impregnated or coated with antimicrobial antibiotics or other wound healing treatment medicinal liquids, creams, gels, oils, compounds, or hemostatic or antihemorrhagic or anesthetic or antiseptic agents/substances or combinations thereof. Likewise, the bandage is to be provided sterile and sealed.

There is shown a complete installation of bandage wherein bandage is placed over the wound of the body member 500. Bandage may be removed and replaced as needed.

The invention may be embodied in other forms without departing from the spirit or essential characteristics thereof. The present embodiment is considered in all respects as illustrative and not restrictive, the scope of the invention being indicated within the meaning and range of equivalency of the claims that are here therefore intended to be embraced.

The embodiments of the invention, as herein described, with references to accompanying drawings to which an exclusive property or privilege is claimed are defined as follows:

1. A bandage of a flexible elastomeric or of like material for the treatment of pressure ulcer wounds (also known as decubitus ulcers or bedsores) and similar ailments, where a bladder body is the primary entity of singular or separated body chambers.
a. The bandage as defined in claim 1, characterized by soft conforming and flexible elastomeric material, or of materials with like properties, that redistributes underlying pressure away from vicinity of the wound to the peripheral of the wound; thus encouraging comfort at wound vicinity.
b. The bandage according to the preceding claims, wherein said bladder recognizes embodiments requiring modification of pressure within the bladder body to accommodate differing body weights and environmental influences during manufacturing and exerted by altitude; and may include fill ports for pressure modification.
c. The bandage as defined in claim 1, characterized by vent openings for encouragement of ventilation at vicinity of a wound.
d. The bladder body according to the proceeding claims, wherein alternative embodiment of independent chambers redistributes pressure to meet varying pressure needs and allowing for bandage movement through shear forces to reduce rubbing or abrasive potential of skin against the bandage or other surfaces.

2. The bandage as defined in claim 1, characterized with a flange or equivalent for the attachment of bandage to human skin, but not defined as necessary for the device to function.
a. The bandage according to the preceding claims, wherein said flange is of like flexible material.
b. The bandage according to the preceding claims, wherein said flange has border for application of an adhesive or other utilities for the attachment to human skin.
c. The bandage according to the preceding claims, wherein said adhesive flange may be protected by a peelable strip (disposable apron).
d. The bandage according to the proceeding claims, wherein said flange has alternative embodiments to include shaped flange tabs for improved connectivity.
e. The bandage according to the proceeding claims, wherein said flange has alternative embodiments to include a thin flexible and breathable flange.

3. The bandage according to any of the preceding claims, where the bandage padding is absorbent by design and air permeable for the allowance of ventilation at wound vicinity, such that the padding textile or equivalently fabricated material can and may conform to contours of the human body at the vicinity of wound.
a. The bandage according to the preceding claims, allows for the padding textile to cover the ports but ventilate through the padding.
b. The bandage according to the preceding claims, allows for the padding textile to remain open at the port openings for increased ventilation past the padding.
c. The bandage according to the preceding claims, wherein said padding textile has alternative embodiments to include quilt design for increased ventilation.
d. The bandage according to the preceding claims, is to be provided sterile and sealed.
